(19) United States
(12) Patent Application Publication
(10) Pub. No.: US 2023/0010507 A1
(43) Pub. Date: Jan. 12, 2023

(54) METHOD FOR PREDICTING AND EVALUATING ADHESION OF COMBUSTION ASH IN COAL-MIXED COMBUSTION BOILER

(71) Applicant: IHI INSPECTION AND INSTRUMENTATION CO., LTD., Tokyo (JP)

(72) Inventors: Kenjiro CHIE, Tokyo (JP); Junichi SHIGETA, Tokyo (JP); Takashi TAKANO, Tokyo (JP); Yoichi NAGASHIMA, Tokyo (JP)

(73) Assignee: IHI INSPECTION AND INSTRUMENTATION CO., LTD., Tokyo (JP)

(21) Appl. No.: 17/945,476

(22) Filed: Sep. 15, 2022

Related U.S. Application Data
(63) Continuation of application No. PCT/JP2021/008132, filed on Mar. 3, 2021.

(30) Foreign Application Priority Data
Mar. 18, 2020 (JP) 2020-048170

(51) Int. Cl. G01N 3/56; G01N 1/38; G01N 1/28; G01N 1/44; G01N 33/22

(52) U.S. Cl. CPC G01N 3/565; G01N 1/38; G01N 1/44; G01N 1/286; G01N 33/222; F23C 2900/01001

(57) ABSTRACT

Provided is a method for predicting and evaluating adhesion of combustion ash in a coal-mixed combustion boiler in which biomass is used as renewable energy, the method comprising: ashing a sample to prepare an ashed test sample, the sample being obtained by mixing the biomass with coal that is main fuel of the coal-mixed combustion boiler, at a predetermined additive ratio; sintering the ashed test sample under a combustion temperature condition of the coal-mixed combustion boiler to generate sintered ash; testing the sintered ash by a rattler tester to obtain a sticking degree from a ratio obtained by dividing a weight of the sintered ash after the test by a weight of the sintered ash before the test; and evaluating in advance an adhesion state of the combustion ash in the coal-mixed combustion boiler on a basis of the sticking degree.

### TECHNICAL FIELD

The present disclosure relates to a method for predicting and evaluating adhesion of combustion ash in a coal-mixed combustion boiler.

### BACKGROUND ART

In recent years, introduction expansion of renewable energy has been an important matter from a perspective of promoting global warming countermeasures, and an increasing number of coal-mixed combustion boilers in which biomass (organic matters derived from animals and plants) is used as renewable energy are utilized in thermal power plants and the like.

Particularly in the case of the thermal power plants, the power generation efficiency is the ratio of energy inputted to a power generation facility and the amount of electric power energy obtained by power generation, and hence the power generation efficiency can be improved by using the biomass that is renewable energy, as the inputted energy.

FIG. 1 illustrates an example of the coal-mixed combustion boiler. In the figure, 1 denotes a boiler body including a furnace 1a formed by a furnace wall tube (heat transmission tube), and a rear heat transfer unit 1b, 2 denotes mixed fuel of coal (pulverized coal) and biomass, the mixed fuel being put into the furnace 1a of the boiler body 1, 3 denotes primary superheaters, 4 denotes a secondary superheater, 5 denotes a tertiary superheater, 6 denotes a final superheater, 7 denotes primary reheaters, 8 denotes a secondary reheater, and 9 denotes economizers. These heat exchangers are constituted by heat transmission tubes.

Then, when the mixed fuel is put and burned in the furnace 1a of the boiler body 1, generated combustion gas heats the heat transmission tube constituting a furnace wall of the furnace 1a, then heats an upper heat transfer unit 11 including the secondary superheater 4, the tertiary superheater 5, the final superheater 6, and the secondary reheater 8 in an upper portion of the furnace 1a, and subsequently heats the primary superheaters 3, the primary reheaters 7, and the economizers 9 in the rear heat transfer unit 1b. Exhaust gas after such heat exchange flows into an exhaust gas duct 10, nitrogen oxides, sulfur oxides, and the like are removed from the exhaust gas by denitration and desulfurization flue-gas treatment devices (not illustrated) provided downstream, and then the resultant gas is discharged to the atmosphere.

Note that Patent Literature 1 given below is an example of prior art document information concerning coal-mixed combustion boilers in which biomass of this type is used as renewable energy.

### CITATION LIST

### Patent Literature

Patent Literature 1: JP 2015-087053A

### SUMMARY

### Technical Problem

However, in the case of such a coal-mixed combustion boiler as described above, depending on the type of biomass used for coal-mixed combustion, fouling (adhesion of combustion ash from the upper portion of the furnace 1a to the heat transmission tubes provided downstream) may occur in the coal-mixed combustion boiler. For example, if such fouling progresses, there is a fear of inducing ash damages such as heat transmission inhibition, clogging, and a physical damage due to a drop of a massive rigid clinker. However, effective measures to evaluate in advance an adhesion state of the combustion ash in the coal-mixed combustion boiler have not existed until now, and it has been difficult to precisely determine how much the additive ratio of the biomass can be increased within the range in which the ash damages are not caused.

The present disclosure has been made in view of the above-mentioned actual circumstance and has an object to make it possible to evaluate in advance an adhesion state of combustion ash in a coal-mixed combustion boiler in which biomass is used as renewable energy.

### Solution to Problem

The present disclosure provides a method for predicting and evaluating adhesion of combustion ash in a coal-mixed combustion boiler in which biomass is used as renewable energy, the method comprising: ashing a sample to prepare an ashed test sample, the sample being obtained by mixing the biomass with coal that is main fuel of the coal-mixed combustion boiler, at a predetermined additive ratio; sintering the ashed test sample under a combustion temperature condition of the coal-mixed combustion boiler to generate sintered ash; testing the sintered ash by a rattler tester to obtain a sticking degree from a ratio obtained by dividing a weight of the sintered ash after the test by a weight of the sintered ash before the test; and evaluating in advance an adhesion state of the combustion ash in the coal-mixed combustion boiler on a basis of the sticking degree.

According to this feature, reproduced is an adhesion state of combustion ash that can occur in the case where coal with which biomass is mixed at the same additive ratio as that of the sample is burned in an actual coal-mixed combustion boiler, and the sticking degree of the combustion ash is used as an index, whereby practical and reliable evaluation can be performed. Therefore, it is possible to easily determine how much the additive ratio of the biomass can be increased within the range in which ash damages are not caused.

Moreover, for carrying out the present disclosure more specifically, an adhesion result of the combustion ash and the sticking degree in the actual coal-mixed combustion boiler may be compared with each other, whereby an adhesion safety margin of the sticking degree may be specified, the adhesion safety margin being a margin in which the ash damages are not caused in the actual coal-mixed combustion boiler. According to this feature, if the additive ratio of the biomass is adjusted such that the sticking degree falls within the adhesion safety margin, the ash damages can be prevented.

Further, in the present disclosure, it is preferable to: obtain the sticking degree for a plurality of ashed test samples among which the additive ratio of the biomass added to the coal is different; and evaluate, as an optimum additive ratio, an additive ratio of the biomass at which the sticking degree has a maximum value within the adhesion safety margin.

Note that, for specifying the adhesion safety margin of the sticking degree in which the ash damages are not caused in the actual coal-mixed combustion boiler, although an occurrence state of the ash damages slightly varies even at the same sticking degree depending on the structure of the actual coal-mixed combustion boiler and the like, if the region in which the sticking degree is equal to or less than 0.5 is specified as the adhesion safety margin, serious ash damages can be regarded as substantially avoidable.

### Effects

According to the above-mentioned method for predicting and evaluating the adhesion of the combustion ash in the coal-mixed combustion boiler of the present disclosure, reproduced is an adhesion state of the combustion ash that will occur in the case where coal with which biomass is mixed at the same additive ratio as that of the sample is burned in an actual coal-mixed combustion boiler, and the sticking degree of the combustion ash is used as an index, whereby practical and reliable evaluation can be performed. Therefore, ash damages such as heat transmission inhibition, clogging, and a physical damage due to a drop of a massive rigid clinker, which are caused if the adhesion of the combustion ash progresses, can be avoided from occurring, and the additive ratio of the biomass can be set as high as possible within the range in which the ash damages are not caused, leading to efficient and effective utilization of the biomass.

### BRIEF DESCRIPTION OF DRAWINGS

FIG. 1 is a schematic diagram illustrating an example of a coal-mixed combustion boiler to which the present disclosure is applied;

FIG. 2 is a schematic diagram of an electric furnace in which sintering of a sample is performed according to a method of the present disclosure;

FIG. 3 is a front view illustrating an example of a rattler tester in which measurement of a sticking degree is performed;

FIG. 4 is a graph illustrating a relation between the sticking degree and a sintering temperature;

FIG. 5 is a graph illustrating a relation between the sticking degree and additive ratios of wood-based biomass materials;

FIG. 6 is a graph illustrating a relation between the sticking degree and additive ratios of other biomass materials;

FIG. 7 is a graph illustrating a relation between an Rf value and the additive ratios of the wood-based biomass materials;

FIG. 8 is a graph illustrating a relation between an Rs value and the additive ratios of the wood-based biomass materials;

FIG. 9 is a graph illustrating a relation between the sticking degree and the Rf value; and

FIG. 10 is a graph illustrating a relation between the sticking degree and the Rs value.

### DESCRIPTION OF EMBODIMENT

Hereinafter, an embodiment of the present disclosure is described with reference to the drawings.

FIG. 2 to FIG. 10 illustrate an example of an embodiment for carrying out the present disclosure. In the present embodiment, a sticking-degree measuring method is utilized, the method being a proven method for evaluating ash dirt (adherability) on low-grade coal (subbituminous coal), and an adhesion state of combustion ash at the time of mixed combustion of coal and biomass is evaluated in advance.

That is, a sticking degree is a degree that is newly defined as an index for quantifying the hardness of a sintered compact by applying a rattler test in which the abrasion resistance and edge stability of a metal green compact are quantitatively evaluated, and a ratio obtained by dividing the weight after the rattler test by the weight before the rattler test is defined as the sticking degree.

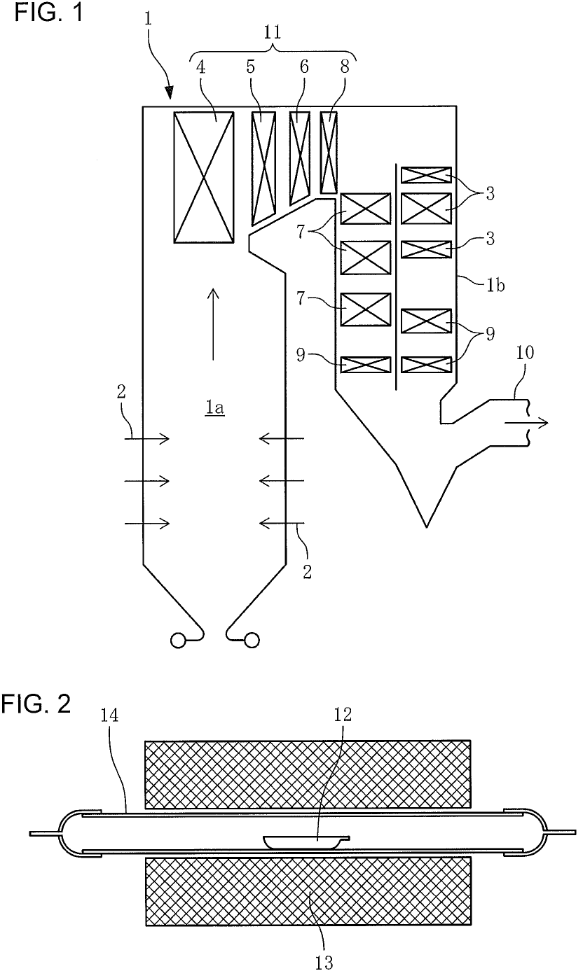

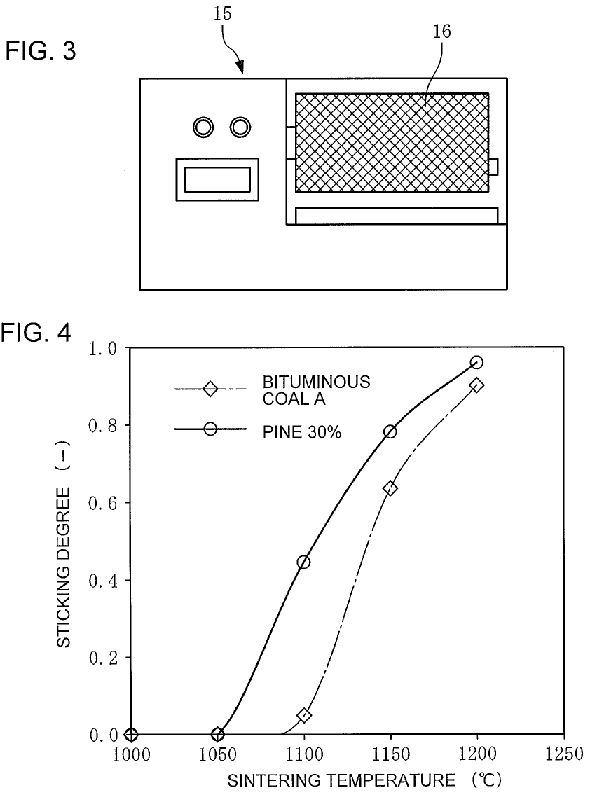

More specific description of the rattler test is given in terms of the present embodiment: a sample is obtained by mixing biomass at a predetermined additive ratio in conformity with JIS (JIS M 8812) that is an analysis method for an ash content of coal; the sample is ashed at 815° C. in a muffle furnace, whereby an ashed test sample is prepared; as illustrated in FIG. 2, the ashed test sample is put on an alumina boat 12, and is then inserted into an alumina tube 14 attached to an electric furnace 13; the ashed test sample is sintered through a heating process under a combustion temperature condition of a coal-mixed combustion boiler (see FIG. 1); and the hardness (the sticking degree to be described later) of sintered ash thus generated is evaluated by measuring using a rattler tester 15 as illustrated in FIG. 3.

Here, the rattler tester 15 is a device for measuring the abrasion resistance and edge stability of a metal green compact, and is a device in which a cylindrical metal mesh 16 (a mesh size of 1 mm#) with a diameter of 100 mm and a length of 120 mm is rotated at a speed of 80 rpm. If the cylindrical metal mesh 16 in the rattler tester 15 is rotated with the sintered ash being put therein, the sintered ash is once lifted upward, then drops and collides against a metal mesh inner wall, and gradually collapses from its surface. Hence, after rotations under given conditions, a sintering property of the ash is evaluated on the basis of the weight of the ash that remains in the cylindrical metal mesh 16.

Note that test conditions can be set, for example, as follows.
Temperature: 900° C. to 1,300° C.
Atmosphere: air
Heating time: 1 hour
Evaluation method: A numerical value obtained according to Equation (1) given below is defined as the sticking degree, and the sintering property is quantified (as the sticking degree becomes closer to 1.0, the ash is sintered harder).
Sticking degree = weight after rattler test / weight before rattler test ... (1)

Table 1

Characteristics of Coal and Wood-based Biomass Materials

| | | | Bituminous coal A | Pine | Cedar | Oak |
|---|---|---|---|---|---|---|
| Higher heating value [MJ/kg] | | Dry basis | 29,010 | 20,490 | 21,170 | 19,500 |
| Total moisture content [%] | | As received basis | 7.3 | 22.9 | - | - |
| Proximate analysis | Moisture content [%] | Equilibrated moisture basis | 2.7 | 12.1 | 15.9 | 15.1 |
| | Ash content [%] | | 14.4 | 0.4 | 0.4 | 0.5 |
| | Volatile matter content [%] | | 27.6 | 74.1 | 68.4 | 72.7 |
| | Fixed carbon[%] | | 55.3 | 13.4 | 15.3 | 11.7 |
| | Fuel ratio [-] | | 2.00 | 0.18 | 0.22 | 0.16 |
| Ultimate analysis | Carbon [%] | Dry basis | 71.4 | 49.9 | 52.3 | 49.2 |
| | Hydrogen [%] | | 4.20 | 6.05 | 6.1 | 6.1 |
| | Nitrogen [%] | | 1.30 | 0.05 | <0.1 | <0.1 |
| | Oxygen [%] | | 7.9 | 43.6 | 41.1 | 44.1 |
| | Total sulfur [%] | | 0.50 | 0.01 | <0.1 | <0.1 |
| | Combustible sulfur [%] | | 0.40 | 0.00 | <0.1 | <0.1 |
| | Incombustible sulfur [%] | | 0.10 | 0.01 | <0.001 | <0.1 |
| Ash composition | SiO₂ | Ash basis | 49.0 | 0.62 | 6.37 | 0.16 |
| | Al₂O₃ | | 32.7 | 0.49 | 1.40 | 0.23 |
| | Fe₂O₃ | | 6.85 | 0.35 | 10.5 | 0.51 |
| | CaO | | 4.62 | 30.8 | 22.1 | 58.5 |
| | MgO | | 0.95 | 10.2 | 8.29 | 9.22 |
| | Na₂O | | 0.30 | 0.52 | 2.17 | 2.94 |
| | K₂O | | 0.40 | 13.6 | 28.9 | 8.82 |
| | SO₃ | | 2.20 | 1.60 | 1.14 | 1.23 |
| | P₂O₅ | | 0.85 | 0.71 | 1.10 | 2.30 |
| | TiO₂ | | 1.35 | 0.01 | 0.20 | <0.01 |
| | MnO | | - | 6.92 | - | - |
| Ash melting point | Deformation temperature [°C] | Oxidizing atmosphere | >1,500 | 1,280 | 1,260 | 1,320 |
| | Softening temperature [°C] | | >1,500 | 1,320 | 1,320 | 1,340 |
| | Hemisphere temperature [°C] | | >1,500 | 1,380 | 1,350 | 1,390 |
| | Flow temperature [°C] | | >1,500 | >1,500 | 1,400 | 1,420 |

Table 1 illustrates characteristic analysis results of coal (bituminous coal) and wood-based biomass materials. As illustrated in Table 1, each of the wood-based biomass materials has such characteristics that the heating value is lower than that of the coal and that the volatile matter content is higher than that of the coal. Because volatile matter combustion is high in combustibility (high in combustion speed), it is considered that the biomass-mixed combustion improves the combustibility.

Moreover, because the nitrogen (N) content and the sulfur (S) content are as low as 0.1% or less, reduction in nitrogen oxides (NOx) and sulfur oxides (SOx) in exhaust gas is predicted in the case of the biomass-mixed combustion. Meanwhile, each of the wood-based biomass materials has such characteristics that the ash content is extremely lower than that of the coal and that the content of potassium oxide (K₂O) that induces ash adherability at high temperature is higher than that of the coal.

FIG. 4 illustrates a relation between the sticking degree and a sintering temperature. As illustrated in FIG. 4, the following was confirmed according to test results obtained by the present inventors: ash was more easily sintered (the sticking degree became higher) by adding a wood-based biomass material to bituminous coal A; and, in the case of adding pine at 30%, a temperature condition under which the same sticking degree as that of the bituminous coal A was obtained was lower in temperature by 30° C. to 100° C.

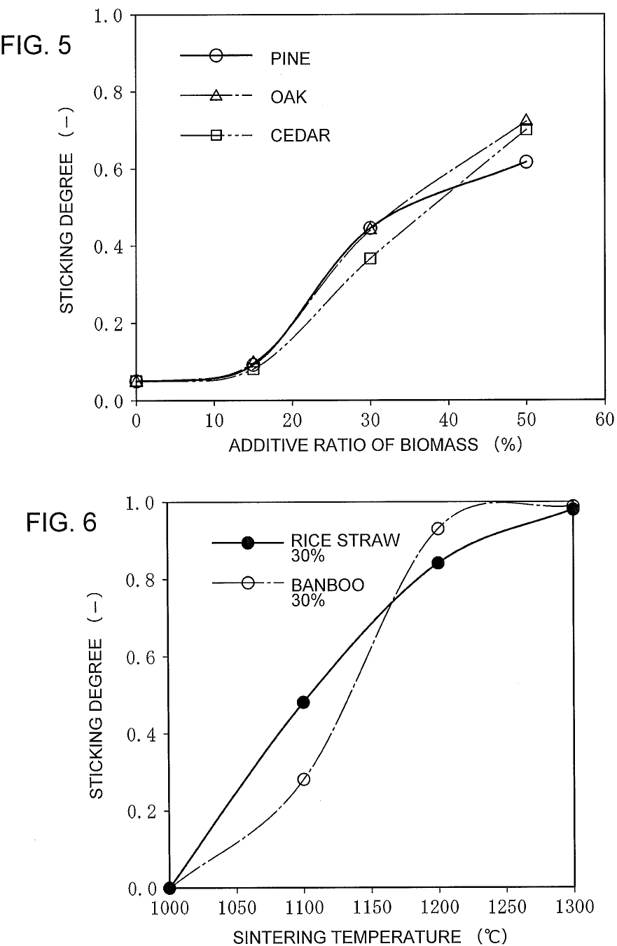

Further, FIG. 5 illustrates a relation between the sticking degree of each of pine, oak, and cedar and the additive ratio of the biomass when a heating temperature of the combustion ash is 1,100° C. As illustrated in FIG. 5, although there was no change depending on a difference in the type of the wood-based biomass material, it was confirmed for all types of the wood-based biomass materials that the sticking degree of the ash became higher as the additive ratio became higher. More specifically, it was confirmed that the sticking degree of the ash rose to 0.6 to 0.7 at an additive ratio of 50%, while the sticking degree of the ash of the bituminous coal A was about 0.05 (see FIG. 4). Note that, if the additive ratio of each wood-based biomass material used this time is equal to or less than about 20%, it is predicted that there is little change in the sintering property of the combustion ash.

Also, for rice straw as an agricultural residue and bamboo as a wood-based material that were selected as other biomass materials, the sticking degree of the combustion ash in the case of mixed combustion at 30% was examined. Characteristics of these biomass materials are illustrated in Table 2, and the sticking degrees thereof are illustrated in FIG. 6. Compared with the sticking degree of the mixed combustion ash of each of the cedar, the oak, and the pine, the mixed combustion ash of the rice straw shows a high value, and the mixed combustion ash of the bamboo shows a low value. Accordingly, it is considered that the sticking degree is influenced by the ash composition that is different depending on the type of biomass.

Table 2

Characteristics of Coal, Rice Straw, and Bamboo

| | | | Bituminous coal B | Rice straw | Bamboo |
|---|---|---|---|---|---|
| Higher heating value [MJ/kg] | | Air dried basis | 29,200 | 16,100 | 19,600 |
| Ash content [%] | | Air dried basis | 11.4 | 11.4 | 0.8 |
| Ash composition | SiO₂ | Ash basis | 57.7 | 67.8 | 19.5 |
| | Al₂O₃ | | 33.0 | 0.27 | 0.36 |
| | Fe₂O₃ | | 3.59 | 0.17 | 0.51 |
| | CaO | | 0.85 | 3.74 | 2.09 |
| | MgO | | 1.46 | 6.70 | 26.1 |
| | Na₂O | | 0.33 | 1.11 | 0.62 |
| | K₂O | | 0.93 | 16.1 | 41.5 |
| | SO₃ | | 0.33 | 1.01 | 2.56 |
| | P₂O₅ | | 0.40 | 2.42 | 2.84 |
| | TiO₂ | | 1.10 | <0.01 | 0.03 |
| | MnO | | 0.05 | 0.49 | 0.19 |

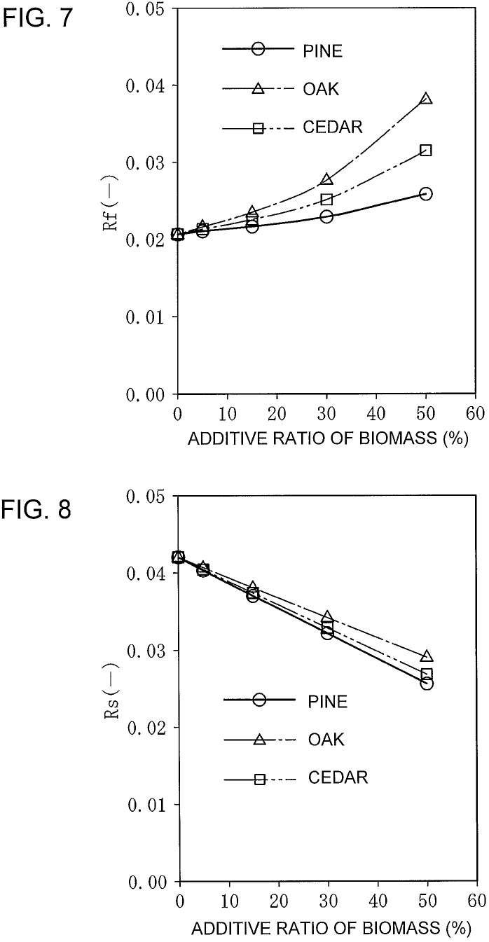

Moreover, how a fouling index Rf and a slagging index Rs of the ash generated by the biomass-mixed combustion change is calculated, and results thereof are illustrated in FIG. 7 and FIG. 8. Note that the unit is the concentration (%) of each component contained in the ash.
Rf = (Base / Acid) × Na₂O ... (2)
Rs = (Base / Acid) × S content of the fuel ... (3)

Here, Base (basic component); Na₂O + K₂O + Fe₂O₃ + CaO + MgO ... (4) Acid (acidic component); Al₂O₃ + SiO₂ + TiO₂ ... (5)

A principal component of each wood-based biomass material is Base. Hence, as illustrated in FIG. 7 and FIG. 8, as the additive ratio is made higher, the Rf value becomes larger, but the Rs value becomes smaller because the wood-based biomass material contains almost no S. As an example, compared with the bituminous coal A, when the additive ratio of the biomass is 50%, the Rf value increases to about 1.5 times, while the Rs value conversely decreases to about ¾. That is, assuming that the conventional indexes can be applied, as a result of adding the biomass, fouling (adhesion of combustion ash from an upper portion of a furnace to heat transmission tubes provided downstream) becomes stronger, and slagging (adhesion of combustion ash in a furnace portion) becomes conversely weaker.

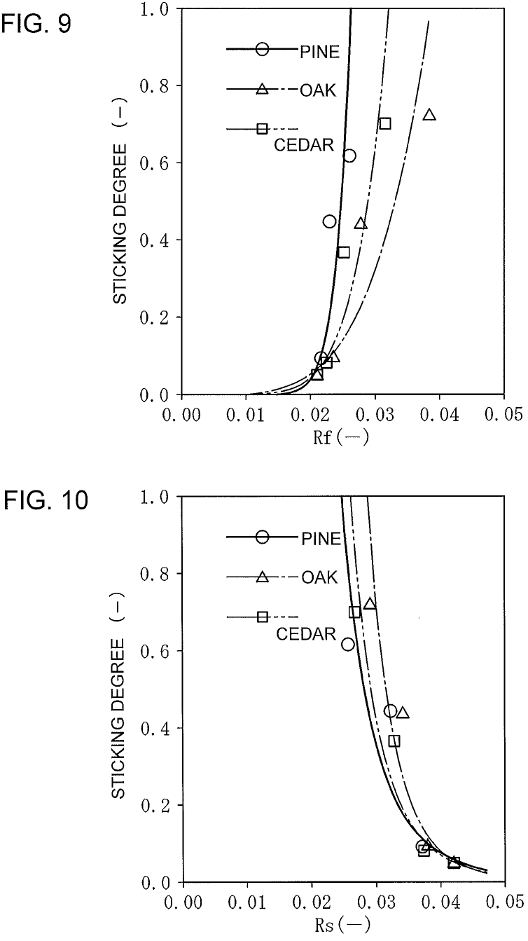

Relations between the sticking degree of the sintered ash obtained by the sintering test and the Rf value and the Rs value are respectively illustrated in FIG. 9 and FIG. 10. As illustrated in FIG. 9 and FIG. 10, it is understood that the sticking degree of the sintered ash becomes higher as the Rf value becomes larger and that the sticking degree of the sintered ash becomes lower as the Rs value becomes larger. On this occasion, the sintering property of the ash and the Rs value have no relevance to each other.

Although the Rs value is proportional to the S content of the fuel as shown in Equation (3) given above, an increase in the S content of the coal is an index for evaluating an influence of the Fe component rather than an influence of the S content, because the Fe component is contained as pyrites (FeS₂) in many cases.

In the coal combustion boiler, a principal factor of the fouling is alkaline components (Na₂O, K₂O) contained in the ash, the ash of each wood-based biomass material contains a particularly large amount of K₂O, and hence the fouling is of greater concern than the slagging. Therefore, it can be said that it is not appropriate to predict an ash damage at the time of biomass-mixed combustion, using the slagging index.

Compared with the sticking degrees of the coal-mixed combustion ash of the pine, the oak, and the cedar, there was a difference in the sticking degrees of the coal-mixed combustion ash of the rice straw and the bamboo.

It is considered that the ash composition of the biomass is greatly related to this result and that the existence of calcium oxide (CaO) and magnesium oxide (MgO) is particularly related thereto.

In general coal ash, CaO is in a compound form of CaSO₄ (CaO + SO₃), and MgO is in a compound form of MgSO₄ (MgO + SO₃) in many cases. On the other hand, because the ash of the biomass contains a small amount of SO₃, there is a possibility that CaO and MgO exist alone. Then, because CaO (melting point: 2,572° C.) and MgO (melting point: 2,852° C.) each have a high melting point, it is considered that CaO and MgO suppress sintering of the ash.

As has been described hereinabove, the ash composition is greatly different depending on the type of biomass, there is a fear that an ash damage occurs depending on the type of biomass and the additive ratio thereof, and hence it is obvious that advance examinations and considerations are necessary. As a result of earnest studies by the present inventors, it was confirmed that the sticking degree of ash was increased by adding biomass to coal and that the sticking degree rose as the mixed combustion ratio increased. Further, a clear correlation between the fouling index and the sticking degree was also confirmed. Therefore, it was verified to be extremely effective to use the sticking degree as an index in order to evaluate the adherability of combustion ash generated at the time of mixed combustion of coal and biomass.

Accordingly, according to the above-mentioned embodiment, reproduced is an adhesion state of combustion ash that will occur in the case where coal with which biomass is mixed at the same additive ratio as that of a sample is burned in an actual coal-mixed combustion boiler, and the sticking degree of the combustion ash is used as an index, whereby practical and reliable evaluation can be performed. Therefore, ash damages such as heat transmission inhibition, clogging, and a physical damage due to a drop of a massive rigid clinker, which are caused if the adhesion of the combustion ash progresses, can be avoided from occurring, and the additive ratio of the biomass can be set as high as possible within the range in which the ash damages are not caused, leading to efficient and effective utilization of the biomass.

More specifically, an adhesion result of the combustion ash and the sticking degree in the actual coal-mixed combustion boiler are compared with each other, whereby an adhesion safety margin of the sticking degree is specified, the adhesion safety margin being a margin in which the ash damages are not caused in the actual coal-mixed combustion boiler. Then, if the additive ratio of the biomass is adjusted such that the sticking degree falls within the adhesion safety margin, the ash damages can be prevented.

Here, for specifying the adhesion safety margin of the sticking degree in which the ash damages are not caused in the actual coal-mixed combustion boiler, although an occurrence state of the ash damages slightly varies even at the same sticking degree depending on the structure of the actual coal-mixed combustion boiler and the like, if the region in which the sticking degree is equal to or less than 0.5 is specified as the adhesion safety margin, serious ash damages can be regarded as substantially avoidable.

This is for the following reason. That is, if the sticking degree is less than 0.2, the ash adhesion state is a powdery state. If the sticking degree is in a range of 0.2 to 0.4, the ash adhesion state is such a state where the ash is fragile and collapses by itself. If the sticking degree is in a range of 0.4 to 0.8, the ash adhesion state is such a state where the ash can be easily collapsed by hand. If the sticking degree is more than 0.8, the ash adhesion state is such a state where the ash melts and firmly adheres in a vitrified state and thus cannot be easily collapsed. These facts are obtained as findings based on past actual-machine adhesion ash examinations. Moreover, it is confirmed that, even if the sticking degree is in the range of 0.4 to 0.8, in which the ash adhesion state is such a state where the ash can be easily collapsed by hand, if the sticking degree is equal to or less than 0.5, serious ash damages are not caused.

Then, the sticking degree is obtained for a plurality of ashed test samples among which the additive ratio of the biomass added to the coal is different, and the additive ratio of the biomass at which the sticking degree has a maximum value within the adhesion safety margin is evaluated as an optimum additive ratio. In this way, the additive ratio of the biomass can be set as high as possible within the range in which the ash damages are not caused, leading to most efficient and effective utilization of the biomass.

Note that the method for predicting and evaluating the adhesion of the combustion ash in the coal-mixed combustion boiler of the present disclosure is not limited only to the above-mentioned embodiment, as a matter of course, the biomass used for mixed combustion in the coal-mixed combustion boiler may be other than the wood-based biomass materials, and various changes can be made within the range not departing from the scope of the present disclosure.

Reference Signs List
1 boiler body
1a furnace
1b rear heat transfer unit
2 mixed fuel
3 primary superheaters
4 secondary superheater
5 tertiary superheater
6 final superheater
7 primary reheaters
8 secondary reheater
9 economizers
10 exhaust gas duct
11 upper heat transfer unit
12 alumina boat
13 electric furnace
14 alumina tube
15 rattler tester
16 cylindrical metal mesh

1. A method for predicting and evaluating adhesion of combustion ash in a coal-mixed combustion boiler in which biomass is used as renewable energy, the method comprising:
ashing a sample to prepare an ashed test sample, the sample being obtained by mixing the biomass with coal that is main fuel of the coal-mixed combustion boiler, at a predetermined additive ratio;
sintering the ashed test sample under a combustion temperature condition of the coal-mixed combustion boiler to generate sintered ash;
testing the sintered ash by a rattler tester to obtain a sticking degree from a ratio obtained by dividing a weight of the sintered ash after the test by a weight of the sintered ash before the test; and
evaluating in advance an adhesion state of the combustion ash in the coal-mixed combustion boiler on a basis of the sticking degree.

2. The method for predicting and evaluating the adhesion of the combustion ash in the coal-mixed combustion boiler according to claim 1, the method further comprising:
comparing an adhesion result of the combustion ash and the sticking degree in an actual coal-mixed combustion boiler with each other; and
specifying an adhesion safety margin of the sticking degree, the adhesion safety margin being a margin in which ash damages are not caused in the actual coal-mixed combustion boiler.

3. The method for predicting and evaluating the adhesion of the combustion ash in the coal-mixed combustion boiler according to claim 2, the method further comprising:
obtaining the sticking degree for a plurality of ashed test samples among which an additive ratio of the biomass added to the coal is different; and
evaluating, as an optimum additive ratio, an additive ratio of the biomass at which the sticking degree has a maximum value within the adhesion safety margin.

4. The method for predicting and evaluating the adhesion of the combustion ash in the coal-mixed combustion boiler according to claim 2, the method further comprising specifying, as the adhesion safety margin, a region in which the sticking degree is equal to or less than 0.5.
